(19) United States
(12) Patent Application Publication
(10) Pub. No.: US 2022/0115115 A1
(43) Pub. Date: Apr. 14, 2022

(54) Popbots: a suite of chatbots to provide personalized support for stress management

(71) Applicant: The Board of Trustees of the Leland Stanford Junior University, Stanford, CA (US)

(72) Inventor: Pablo Enrique Paredes Castro, Palo Alto, CA (US)

(21) Appl. No.: 17/500,880

(22) Filed: Oct. 13, 2021

Related U.S. Application Data
(60) Provisional application No. 63/091,739, filed on Oct. 14, 2020.

(51) Int. Cl. G16H 20/70; G06F 9/451; H04L 12/58

(52) U.S. Cl. CPC G16H 20/70; H04L 51/02; G06F 9/453

(57) ABSTRACT

A method of using chatbots defined as digital services for personalized support for stress management is provided. A chatbot stimulates conversation with human users. A suite of chatbots is a group of functionally and broadly domain-related multiple chatbots, which nevertheless have different identities and/or different knowledge sub-domains which are presented to the user. The specific stress management addressed herein is related to daily stressors which are defined as daily acute issues such as deadlines or difficult social interactions that can generate perceived stress whose intensity can range from mild to severe. As a routine part of everyday life, these stressors are normally repetitive, and their negative effects can be cumulative if they are not appropriately dealt with in a timely way. The method of using chatbots delivers micro-interventions which lower barriers in time and commitment for users in daily stress management.

### CROSS-REFERENCE TO RELATED APPLICATIONS

This application claims priority from U.S. Provisional Patent Application 63/091,739 filed Oct. 14, 2020, which is incorporated herein by reference.

### FIELD OF THE INVENTION

This invention relates to chatbots for personal support, in particular stress management.

### BACKGROUND OF THE INVENTION

In the US approximately 60-80% of primary care visits have a psychological stress component, but only 3% receive stress management advice. The reason for this is a combination of both limited infrastructure geared towards preventative health and limited focus on stress management. However, the increasing accessibility of mobile computing has spurred the growth of mental health applications, which currently account for 29% of the mobile health application market that includes fitness, nutrition, and other lifestyle applications.

Despite the popularity of single-purpose mobile apps, general trends demonstrate that consumers are spending considerably more time with messaging services. As a result, developers are leveraging these messenger clients to build conversational interfaces, also known as “chatbots”, to create new interactions in the health domain including allowing users to report symptoms, make appointments, and gain referrals.

Advances in Natural Language Processing (NLP), such as intent or emotional recognition based on very large language datasets, continues to increase the range of these systems and their potential for impact. Research into improving conversational systems spans a number of domains such as customer service, companionship and, increasingly, mental.

As chatbots are scalable and easy to access, many systems are aimed at substituting human support in common conversations with known formats. Early efforts in mental health include Eliza which attempted to model the psychoanalytical approach of introspection; asking questions to engage the user examining their own mental and emotional processes. More recently chatbots have been used to provide Cognitive Behavioral Therapy (CBT) support to people with risk for depression. However, given the complexity of life and the many types of stressors that a chatbot would need to understand to provide support, building a proactive everyday stress management chatbot that addresses the thousands of known stressors is complex to design, costly to develop, costly to maintain and modify and difficult to author in ways that will appeal universally and broadly to individual preferences.

The present invention addresses these limitations.

### SUMMARY OF THE INVENTION

### Definitions

A chatbot (short for chatterbot, and also referred herein as a digital service) is defined as a computer program designed to stimulate conversation with human users. It has a computer user interface that displays the chatbot's identity (e.g. image, name, and/or style), and an artificial intelligence (AI) based or rule-based backend computer service that determines the knowledge domain, i.e. the range of options that a chatbot can answer.
A suite of chatbots (also referred to as a plurality of digital services) is defined as a group of functionally and broadly domain-related multiple chatbots, which nevertheless have different identities and/or different knowledge sub-domains which are presented to the user.
A shallow (stress management interventional) chatbot is defined as a single-function chatbot that uses few (between 10 to 20) and brief (a few words or choices) conversational exchanges (to deliver a single coping technique for daily stressors).
Daily stressors are defined as daily acute issues such as deadlines or difficult social interactions that can generate perceived stress whose intensity can range from mild to severe. As a routine part of everyday life, these stressors are normally repetitive, and their negative effects can be cumulative if they are not appropriately dealt with in a timely way.
User interaction with a suite of chatbots is defined as methods by which a user interfaces with different chatbots for related purposes (e.g. stress management), as opposed to only interfacing with a single chatbot all the time for many purposes. A single chatbot could cover a larger knowledge domain, having the functions, but not the personalities of multiple shallow chatbots, but cannot replace or achieve the potential engagement effects of multiple identities, generating a different type of perception and therefore different interaction with the user.

The present invention provides a method for the personalized management of daily stressors. A plurality of digital services operates on a computer platform. In one example, the computer platform is a computer, a laptop, a smart phone, a computer tablet, a vehicle digital information system, a smart watch, a smart speaker, or an interactive computing device designed for user interactions. In another example, the computer platform is one or more computer servers and/or cloud services operating via an Internet protocol and communicating for user interaction with a computer, a laptop, a smart phone, a computer tablet, a vehicle digital information system, a smart watch, a smart speaker, or an interactive computing device designed for user interactions.

The plurality of digital services can be displayed on the graphical computer user interface as multimedia elements including icons, images, text or a combination thereof.

The plurality of digital services is displayed on a graphical computer user interface to a user. Each of these digital services uses natural language for interacting with the user and is a script uniquely focusing on a single coping technique or type of intervention for daily stressors. In one example, each script is a pre-scripted set of open conversational exchanges each containing 10 to 20 conversational exchanges and the script lasting about 2 to 3 minutes total.

The method further includes the step of selecting one or more digital services from the plurality of digital services for interfacing and interacting with the user via the graphical computer user interface. The selected one or more digital services are related types of coping techniques or interventions that are based on an initial input received from the user via the graphical computer user interface.

In one example, the step of selecting one or more digital services is done randomly by a software program running on the computer platform. In another example, the step of selecting one or more digital services is done randomly by a software program running on the computer platform based on a condition or user input. In one example of an added capability of the method, the user is capable of requesting a change in the selection of the one or more digital services.

The user interacts with the graphical computer user interface by text, speech, or manipulating interface menus, buttons or multimedia interactive elements.

The method then further includes the step of conducting with the user, via the computer user interface, the open conversational exchange with the user based on one or more selected digital services.

The method could further include steps of assigning and labeling a daily stressor based on the conversational exchange.

The advantages of creating multiple shallow chatbots are manifold:
(i) chatbots capable of delivering specialized micro-interventions lower barriers in time and commitment for users,
(ii) chatbots can be authored and curated more quickly by novice designers to produce a variety of high-quality advice options,
(iii) the variety of chatbots could help improve long-term engagement (i.e., chatbots that “fail” could be more easily modified, removed, or substituted), and
(iv) a suite approach allows for ongoing personalization, wherein reinforcement learning, and other artificial intelligence models can be used to determine and deliver interventions based on user needs in the moment as well as personal preferences for interactions with different chatbots to promote adherence.

### BRIEF DESCRIPTION OF THE DRAWINGS

FIG. 1A shows according to an exemplary embodiment of the invention a system diagram that links the front-end interaction with the user via the Telegram Messaging platform, the database where we maintain information about the user and the use of the chatbots, and the series of chatbot scripts.

FIG. 1B shows according to an exemplary embodiment of the invention a user initiating a conversation over the Telegram interface being asked to describe the stress, a response made by the database to identify the type of stress, and a salute from the multiple bots.

FIG. 1C shows according to an exemplary embodiment of the invention a sample conversation with Doom-bot recommended by the system.

FIG. 2 shows according to an exemplary embodiment of the invention in-situ effectiveness of individual chatbots.

FIG. 3 shows according to an exemplary embodiment of the invention stressor assignments by resource in card sorting task.

### DETAILED DESCRIPTION

To address the limitations in the art the inventor explored the creation of a new breed of short and simple conversational chatbots for in-the-moment management of daily stressors (e.g., deadlines, difficult social interactions, lack of sleep).

An objective for this creation was to create “shallow”, yet effective and engaging mental health chatbots that do not try to replicate human intelligence. In the context of daily stress management, the inventor defined shallow chatbots as those that use few and brief conversational exchanges to deliver a single coping technique as defined supra. These shallow chatbots are not created to replicate or replace humans (i.e., family, friends, or therapists), but rather to operate as part of a larger ecosystem of agents providing stress management support.

The present invention finds basis in the research on micro-interventions and expands that by exploring a suite of diverse and specialized shallow chatbots for daily stress that is herein called PopBots. The name PopBots was selected for several reasons, but primarily for the euphonious connotation that the chatbots might “pop up” for the user at moments of need, and also because it references to population health, which is the basis of the approach taken by this technology, which aims to provide support to broad populations. In suites of shallow chatbots, the questions are exploratory and include: How might one design multiple shallow chatbots for both proactive and reactive stress management? How might everyday users react to using these multiple chatbots for managing their daily stress? And, what challenges and benefits do they perceive about such systems?

To begin answering these questions, the inventor recruited everyday participants from a University community to explore the use of a suite of micro-intervention chatbots for daily stress management. Here the inventor reports on results from two preliminary studies: (i) a three-day, lab-based Wizard of Oz (WoZ) study with N=14 participants and (ii) a one-week, online pilot study with N=47 participants. Results from the formative WoZ study, which compared single and multiple chatbot conditions, suggest that the availability of multiple chatbots might be more effective in the long term with respect to reducing perceived stress compared to a single chatbot. Building on this work, the main study findings highlight users of the online chatbot suite tended to:
(i) see a decrease in depression symptoms as indicated by their PHQ-4 scores,
(ii) view conversations as Helpful to Neutral overall for managing daily stress, and
(iii) come away with increasingly positive sentiment toward the use of chatbots for supporting stress management.

Noted across both studies participants perceived value in having access to a suite of chatbots for stress management. Moreover, follow-up interviews with a subset of participants suggest that almost half of common daily stressors could be discussed with chatbots, potentially reducing the burden on more expensive and scarcer human mental health coping resources. In particular, there is a desire among users to have access to these chatbots for coping with low complexity stressors (i.e., practical and day-to-day concerns) versus high complexity stressors (i.e., those of a more social or interpersonal nature) due to the relative ease of accessing chatbots, the perception of privacy granted by such systems compared to human coping resources, and as a way of avoiding having to place additional burden on friends and family who provide regular support.

The discussion focuses on similarities and differences across these two studies as well as the implications for the design of similar systems. As a result, this study contributes to:
(i) the design and evaluation of a novel suite of shallow chatbots for daily stress management using random assignment,
(ii) a summary of benefits and challenges associated with such systems, and
(iii) design guidelines and directions for other/future research into similar shallow chatbots and micro-interventions suites.

The following: (i) provides background on daily stress as well as traditional mitigation techniques and (ii) describes the state of the art in terms of research on micro-interventions, chatbots, and chatbots for mental health.

### Daily Stress

The stress response is an evolutionary mechanism that mobilizes bodily resources to help humans cope with daily challenges as well as life-threatening situations. Stress has two components, a stressor and a stress response. The former could be linked to sources of uncertainty, complexity, cognitive loads, or emotional distress.

The latter is the mental and physical reaction to such stimuli. Daily stressors are defined as the routine challenges of day-to-day living. The challenges can either be predictable (e.g. daily commutes) or unpredictable (e.g. an unexpected work deadline) and occurs in 40% of all days. Unlike chronic stress, these stressors are relatively short-lived and do not persist from day to day. However, daily stress has been shown to exacerbate symptoms of existing physical health conditions. Repeated triggering of daily stress can also lead to chronic stress, which has been associated with a variety of patho-physiological risks such as cardiovascular diseases and immune deficiencies—conditions that impair the quality of life and shorten life expectancy. Thus, having effective mitigation strategies for daily stress can have a positive effect on a person's wellbeing and overall health.

### Traditional Stress Mitigating Interventions

There are a wide variety of methods employed to help reduce stress. Positive psychology, for instance, is an emerging practice to help people calm down with personally targeted cues, such as asking people to express gratitude or perform compassionate acts. Another group of effective techniques are part of Cognitive Behavioral Therapy (CBT) which teaches people how to recognize their sources of stress, change their negative behavioral reactions, and reframe their thoughts. Yet another approach is the use of Narrative Therapy which focuses on constructing conversations to help people become satisfied with their state of being. Such conversations are the basis of social interaction which has a direct impact on emotions. For example, positive social interactions have been shown to lead to calmness and openness in social engagement. In this invention, the inventor designed chatbots to guide users through stress-relieving techniques in response to daily stressors.

The technology disclosed herein is unique as it:
(1) Transcends traditional methods which have historically relied on the availability of a human professional for their delivery.
(2) Specifically, does not discount or replace these interventions, but rather integrates them into a new delivery system with the benefits described herein.

### Stress Mitigating Micro-Interventions

A relevant approach to this invention is the use of technology that leverages specific CBT techniques (e.g., smoking cessation) to deliver personalized treatments. Recently, researchers have started to explore the use of machine learning algorithms to recommend calming interactions with web apps. For instance, the inventor has demonstrated the benefit of using just-in-time web-based interventions for teaching long-term stress coping skills. In particular, the authors discussed the importance and complexity of engaging people to avoid early attrition. People under high levels of stress find that any additional task, including interventions, adds to their stress load. This motivates the need for the development of designs of intervention suites that could reduce attrition by diversifying the types of interventions that are recommended to users over time.

### Chatbots

Chatbots are digital services that use natural language as the primary means of user interaction. Chatbots are often accessible through common communication platforms such as Facebook Messenger and Twitter. The services are accessed through the same interfaces as human contacts (i.e., to use a chatbot a person simply adds it to their contacts and starts chatting). As a result, the experience is immediate, intuitive, and similar to what they are already doing in the messaging app, chatting with others.

Various types of chatbots exist and most can be categorized along a simple continuum of conversational fluency. At one extreme are bots that respond to any user input allowing for open-ended conversations. This is convenient for the user as the chatbots mirror how they typically use messenger platforms. However, as chatbots are still in their early stages of development they can be conversationally clumsy at times, can fail to recognize certain requests, and may not respond appropriately. At the other extreme are chatbots that adhere to tightly scripted conversations. These yield predictable and stable user interactions but are limited in their conversational scope. Many of today's chatbots fall somewhere in the middle, incorporating aspects of both scripted and open-ended conversations. By design, the chatbots of this invention allow for open-text conversations while primarily relying on scripted conversations with a limited scope (i.e., communicating a coping technique to a user) while allowing for some deviations in response (e.g., branches to handle yes/no responses).

### Chatbots for Mental Health

Chatbots have a long history of application in mental health. The earliest mental health chatbot, ELIZA, was programmed to deliver non-directive therapy mirroring Rogerian therapy (i.e., reflecting and rephrasing user input). Although ELIZA was developed in the 1960s, work on subsequent mental health chatbots has not emerged until recently. A few years after ELIZA's introduction, PARRY was used to study schizophrenia. In addition to regular expressions, PARRY included a model of its own mental state, with affect state. For example, PARRY could become more angry or mistrustful, thus generating “hostile” outputs. In a comparison study, psychiatrists could not distinguish transcripts of interviews with PARRY from those of people with schizophrenia.

Two recent examples are Woebot and Wysa. Woebot is an automated chatbot based on principles of CBT. Woebot leads users through a series of CBT-type lessons, directing users to videos and other forms of didactic material to get them to engage in common CBT skills such as cognitive restructuring or behavioral activation. Wysa is an artificial intelligence driven self-styled “pocket penguin” that also bases chat interactions on CBT skills. The benefits of Woebot have been demonstrated in a randomized controlled trial showing superiority to a web-based eBook at reducing symptoms of depression and anxiety in a college student sample. While most mental health chatbots are aimed at improving wellbeing, to the inventor's understanding none specifically focus on daily stress management. Moreover, this expanding ecosystem of applications suggests chatbots are having an increasing impact on work in digital mental health. This is not surprising given that mental health has long relied on the “talking cure” as a primary form of treatment. One challenge from existing chatbot systems is the need to explore the problems through a set of questions and answers and conversational exchanges that may be hard to author and maintain. The system of this invention overcomes this limitation by allowing for the creation of multiple chatbots that each represent a single type of intervention. Authoring these “shallow” bots is easier for a designer because they can focus only on delivering a single intervention technique with a clear objective and end. For users, micro-intervention chatbots offer quick advice without needing to work through a lengthy dialog which could be, by itself, another source of stress. In some ways, the system of this invention resembles a “game console” or a media platform (i.e., Netflix) where each chatbot is a new “game” or “movie” and the authors of the system can learn over time which people prefer.

Furthermore, the authoring, development, quality assurance, and testing of these shallow chatbots is significantly easier and more reliable than complex chatbots.

### Method

The following describes the design process behind the chatbot suite of this invention before detailing the online study protocol.

### Prototype Chatbot Suite

Extending on micro-interventions to conversational interfaces, the inventor developed the creation of a suite of shallow chatbots that provide in-the-moment conversations for managing daily stress. While prior work tends to focus on patients or people at clinical risk (i.e., people with high symptom levels of depression or anxiety, the aim in this invention was to provide a quick and engaging system using short micro-intervention chatbots that can help to alleviate daily stress for healthy people (i.e., toward improving long-term wellbeing and/or helping to mitigate future crises). Another goal for this invention was to simplify authoring of chatbots by reducing complexity toward enabling a scalable solution for rapidly creating numerous (i.e., hundreds or more) chatbots for stress management. To those objectives, the inventor developed and here describes an exemplary chatbot suite with a common template for short conversations (i.e., 2-3 minutes with a few conversational exchanges) composed of several components:
(i) an onboarding script for explaining the system and its limitations to users,
(ii) a shared set of greetings, stressor parsers, and intent extraction components, and
(iii) micro-intervention chatbots that make up the suite, and
(iv) a feedback component.

### Chatbot Design Approach

An iterative, human-centered approach was used to design the chatbot suite (e.g., Table 1). Initial chatbots scripts were developed in a 4-hour workshop with the aid of 6 designers, curated by a clinical psychologist, and tested for quality purposes by conducting simulations where pairs of designers acted as user and chatbot. Each chatbot relied on a decision tree to facilitate conversations, usually resulting in the user providing a response to a series of open-ended (e.g., what is the worst case scenario for [a stressor]?), yes/no (e.g., has [the stressor] affected your sleep?), or numerical (e.g., what is the severity of a scenario?) questions (e.g., Table 2). Stress management literature, particularly literature related to Cognitive Behavioral Therapy (CBT) techniques, was used to derive conversations for stress relief. Using this approach, the design team created chatbots based on 4 techniques: Positive Psychology, CBT, Somatic Relaxation, and Meta-cognitive Relaxation. The total development time (i.e., including design, curation, and quality assurance steps) was about 8 hours.

TABLE 1
Prototype chatbot names, their techniques,
and which studies they were used in.

| Chatbot (-bot) | Technique | Description | Study |
|---|---|---|---|
| Doom | Worst-case | Ask the user to consider the | Both |
| | Scenario | worst-case scenario. | |
| Sherlock | Problem | Asks a series of questions to | Both |
| | Solving | pin-point the problem. | |
| Glass-half-full | Positive | Lets the user view their | Both |
| | thinking | problems in a new light. | |
| Sir Laughs-a | Humor | Finding humor in the situation | Online |
| Treat yourself | Self-love | Letting the user know it is | Online |
| | | alright to treat themselves. | |
| Dunno | Distraction | Asks user to think about events | Online |
| | | they are looking forward to. | |
| Checkin | Checking in | Asks whether the stressor | Online |
| | | affected daily activities. | |

### Formative Study

To explore the feasibility of the suite of chatbots, the inventor first conducted a Wizard of Oz (WoZ) formative study with follow-up interviews. This study used a subset of chatbots from Table 1 and allowed the inventor to explore:
(i) initial reactions to using suites of chatbots versus singular chatbot apps, and
(ii) the types of stressors, if any, users would be willing to talk to chatbots about.

The study lasted 3-days with participants meeting with an experimenter daily for in-person sessions. N=14 participants were recruited (7 male, 6 female, 1 non-binary; age range 18-50) via university listserv. Most ( 13/14) were university students while one was a staff member.

### Study Protocol

In one example a web chat interface (tkl) was used that allows the creation of open chat channels, to serve as the interface between participants and experimenter. Participants believed they were interacting with a chatbot while in reality they were interacting with the experimenter who was following the conversational scripts created in a workshop beforehand (e.g., Table 2). Each participant was randomly assigned to either a Variable condition that had three chatbots (i.e., Positive Thinking, Worst Case Scenario, and Problem Solving) or a Control condition that contained only the Problem Solving chatbot. Participants in the Variable condition were matched with different chatbots during each session using Latin Squares Randomization. During each session, participants had a single conversation. The participants were instructed to type a greeting (e.g., “Hi”) in the chat channel which cued the experimenter to start following the script. After 3 sessions, participants completed a post-study questionnaire about perceived efficacy in stress reduction and usability of the chatbots. Four participants were contacted for semi-structured interviews—two from each

TABLE 2
Example chatbot script
Doom-bot

Tell me more details about [problem]?
I'm sorry to hear that. What are you most afraid might happen as a result?
Alright, on a scale of 1 to 10, 1 being impossible, 10 being certain,
how likely is this scenario?
Alright, in the case that this happens, what could you do to get back
on track?
Cool, looks like you have a plan B. Just remember, though you cannot
control everything, there is a way to get back on your feet

experiment condition. Each pair of individuals was made of one individual who evaluated the chatbot as effective and one that did not find the chatbot as effective. Each of those users at the variety of stressors into buckets (i.e., chatbots and their coping strategies) while using a “Think Aloud” protocol to understand trends and underlying motivations. Protocols were reviewed for ethics and privacy concerns by the institution's Research Compliance Office.

### Study Results

Here the results from our preliminary study are briefly summarized with respect to perceived stress reduction and overall impressions of the chatbots by participants. As participants in the later studies completed the same interview and card-sorting activities, these results will be discussed in more detail infra.

Perceived Stress Reduction. When asked about their interactions with the chatbots, analysis of the data shows differences in self-reported stress between conditions. Participants reported a higher perception of stress reduction in the Variable chatbot condition (blue, left is better) which helps motivate our approach to designing suites of chatbots for daily stress.

Perceptions of Chatbots. Overall, participants who completed follow-up interviews were positive about chatbot systems. Most (¾) were interested in using chatbots for coping with daily stressors even when support from humans was available. The objectivity, ease of use, and privacy chatbots offered compared to human conversational partners was appealing for situations like illness and injury, financial problems, and social isolation. Participants believed that the chatbots would be effective because they provided quick therapy solutions on the spot. For example, one participant stated, “I'd rather talk about these [problems] in the void . . . and have a computer interact with me quickly”. Another participant preferred to talk to humans. That being stated, all participants expected chatbots to have human-like characteristics (e.g., a typing delay despite being aware that chatbots can respond faster), corroborating prior work on the mirroring of non-verbal, conversational, and personality cues. Additionally, one participant from the Variable condition described using multiple chatbots (i.e., multiple micro-interventions) in sequence to help with finding appropriate solutions for complex stressors (e.g., using Positive Thinking to reduce anxiety first and then Problem Solving to take care of the underlying problem) which was not possible in the control condition where participants interacted with a single chatbot (i.e., Problem Solving) providing one intervention.

### Main Study

To evaluate the suite of chatbots, the inventor conducted a rolling field study with students and staff from our university community. This study used all the chatbots from Table 1 and allowed further exploration of potential efficacy, perceptions of the suite approach to chatbots for daily stress management, and the types of stressors users might be willing to discuss with such systems.

### System Implementation

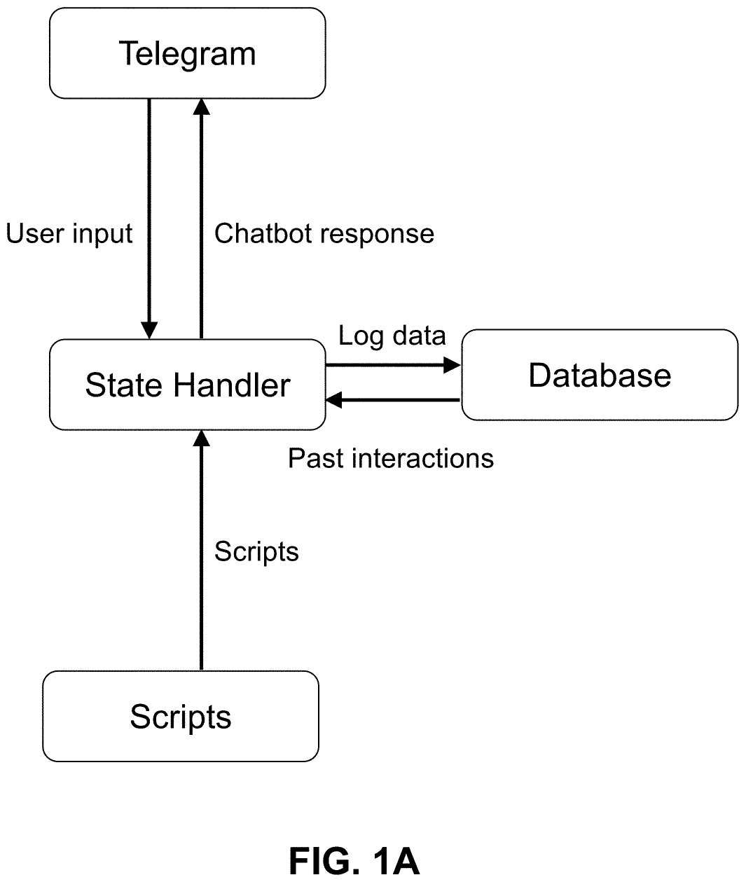

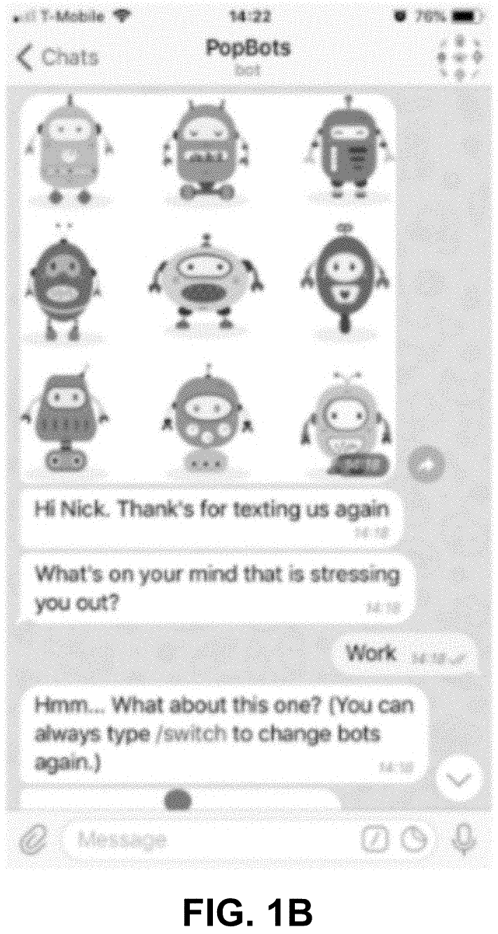

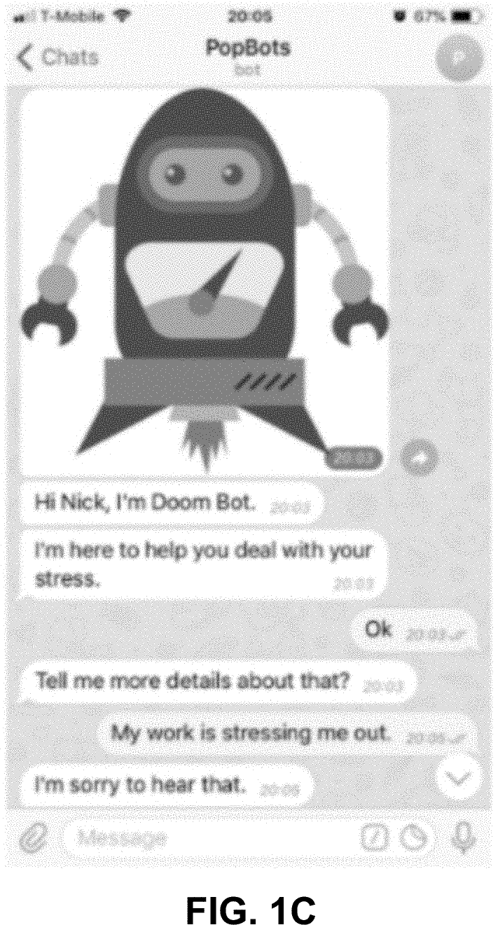

Based on the results from the formative study, the inventor implemented the chatbot suite in Telegram, a data-security compliant messaging platform, using a Python backend and a MongoDB database (FIG. 1A). Using prior experience and observations of the initial chatbot workshop, the inventor generated four additional chatbots bringing the system total to seven and programmed the conversational scripts in Python. Interactions with these chatbots are automatic and rule-based, using regular expressions to control the flow of conversations. Following the conversational template, when the user messages the chatbots (i.e., by typing “Hi”) they receive a friendly greeting message and are asked to describe their current stressor (FIG. 1B). After extracting the stressor, a chatbot is randomly recommended (FIG. 1C). User responses are passed to a state handler via the Telegram API; the state handler analyzes the responses to generate a response. Once the response is generated it is sent to the user and the interaction is logged. After the conversation ends, the chatbot thanks the user for sharing and asks them for feedback on whether the interaction helped to reduce their stress on a 3-point scale (i.e., “Helpful”, “Neutral”, and “Not Helpful”). The inventors refined the chatbots with pilot users to make them appear more human-like (e.g., introducing typing delays), clarified utterances so users were more aware of when the system was waiting for input, and added a “/switch” option that allowed users to change chatbots in-situ.

### Protocol

Participants were recruited and recruitment materials specified that participants would be asked to use our system for 7 days and complete a pre-study questionnaire, short daily surveys, and a post-study questionnaire. These materials also specified that participants had to be 18 years of age or older and have a compatible smartphone (i.e., Android, iPhone). Online enrollment occurred on a rolling basis and all questionnaires were completed via the Qualtrics survey tool.

After receiving the invitation email, participants could complete a pre-study questionnaire which asked them about their demographic information, how much stress they felt daily, and their perceptions of using chatbots for daily stress management. Participants also completed the short Patient Health Questionnaire (PHQ-4) to ascertain a measure of clinical anxiety and depression symptoms [29]. Upon completing the survey, participants were automatically sent email instructions for installing the Telegram application as well as a personalized URL which, when accessed on their smartphones, initialized the Popbots channel within the application.

Once this initialization was completed, participants were instructed to type “Hi” and go through the onboarding script, which explained the purpose of the system (e.g., that it was for daily stress management) and its limitations (e.g., that it was not intended for the treatment of serious mental health conditions). At the end of the onboarding script, participants were instructed to interact with the chatbots anytime they felt stressed over the next 7 days. Daily surveys were sent at 8 pm each evening (local time) and it asked participants to rate their daily stress levels, sleep quality the previous night, and level of social interaction experienced that day. After seven days of using the system, participants completed a post-study questionnaire. The post-study questionnaire asked participants about their perceptions of daily stress over the course of the week, if their perceptions of chatbots had changed, and other usability questions. Participants also again completed the PHQ-4 questionnaire. The inventor followed up with a subset of participants to complete the same semi-structured interview and card sorting task; a general email request was sent to all participants and volunteers were enrolled on a first-come, first-served basis.

### Data and Analysis

In summary, the data includes responses to pre-, daily, and post-study questionnaires, conversational logs from the chatbot system, interview transcripts, and photographs of assignments made during the card-sorting activity. All questionnaires include Likert scale question and short open-form responses. As exploratory work, descriptive statistics are reported and includes trends in this data, which are contextualized with participant quotes. Follow-up interviews were audio recorded, transcribed, and coded for themes of interest. An iterative analysis approach was pursued using a mixture of inductive and deductive codes. A code book was created initially derived from research literature, the study protocol, and post-interview discussions amongst the research team. The unit of analysis was an answer (or stream of answers) to specific questions. High-level codes included perceptions of chatbots for stress managements, preferences around conversational partners, as well as privacy and trust. A random transcript was selected and co-coded by the research team. Remaining transcripts were divided and coded independently. The individually coded transcripts were then reviewed by a second researcher who met with the original to resolve disagreements. Two researchers then aggregated transcripts, reviewed for consistency, and summarized results.

TABLE 3
Categories of Stressors.

| Stressor | Count (%) | |
|---|---|---|
| Work, School, & Productivity | 79 | (40%) |
| Health, Fatigue, & Physical Pain | 27 | (13%) |
| Social Relationships | 21 | (10%) |
| Financial Problems | 13 | (6%) |
| Emotional Turmoil | 12 | (6%) |
| Family Issues | 10 | (5%) |
| Everyday Decision Making | 8 | (4%) |
| Other | 27 | (13%) |

| | |
|---|---|
| Total | 197 |

### Participants

N=47 participants were recruited (34 female, 13 male, 0 non-binary). Participants were between 18-24 years old.

### Results

While 47 participants enrolled in the study, 31 (69.5%) completed both the pre- and post-study questionnaires. Descriptive statistics was reported such as means (M=X) and standard deviation (SD=X).‘P’ and randomized IDs was yused to refer directly to participants in the online study (e.g., P1234) and letters (e.g., PX) to refer to participants in a prior WoZ study.

### Application Logs

Over the course of 7 days, most participants ( 44/47) interacted with our chatbots generating 291 conversations. Participants averaged about seven conversations per week (M=6.83, SD=3.14). These conversations were short, lasting only a few minutes (M=1.95, SD=2.53) minutes, and often occurred in the later part of the day. While some conversations were likely triggered by the daily survey reminder (at 8 pm), the vast majority (80%) of conversations were unprompted and occurred throughout the day with increased activity in the 7 am, 12 pm, 3 pm, and 8 pm hours. A deeper exploration of these conversations indicated that some participants were simply checking in, particularly around 8 pm, reporting stressors such as “nothing” or “doing pretty good actually”. As a result, about a third of conversations was filtered out that fell into this category or those that contained a technical issue making them indecipherable.

### Reporting Stressors

Two ways were observed that participants reported stressors to the chatbots. Most participants (74%) tended to describe stressors in a few words. For example, participants wrote “Having to go to work tomorrow”, “My presentation that's coming up”, and “My friend being mad at me”. Another common approach (26%) was to type out single keywords (e.g., “money”, “car”, “family”).

### Topics of Conversation

After filtering, 197 conversations were labeled using 8 category tags representing consistent topics that participants discussed with the chatbots (Table 3). The most common topics included:
(i) work and school related productivity issues,
(ii) health problems (e.g., feeling tired, experiencing pain), and
(iii) interpersonal issues related to (non-familial) social relationships.
There was also a number of “Other” conversations that were not widely discussed, but might point to additional topics of daily stress including: vacation-related stress (e.g., packing), commuting, and seasonal stressors (e.g., holiday-related gift giving).

### In-Situ Efficacy

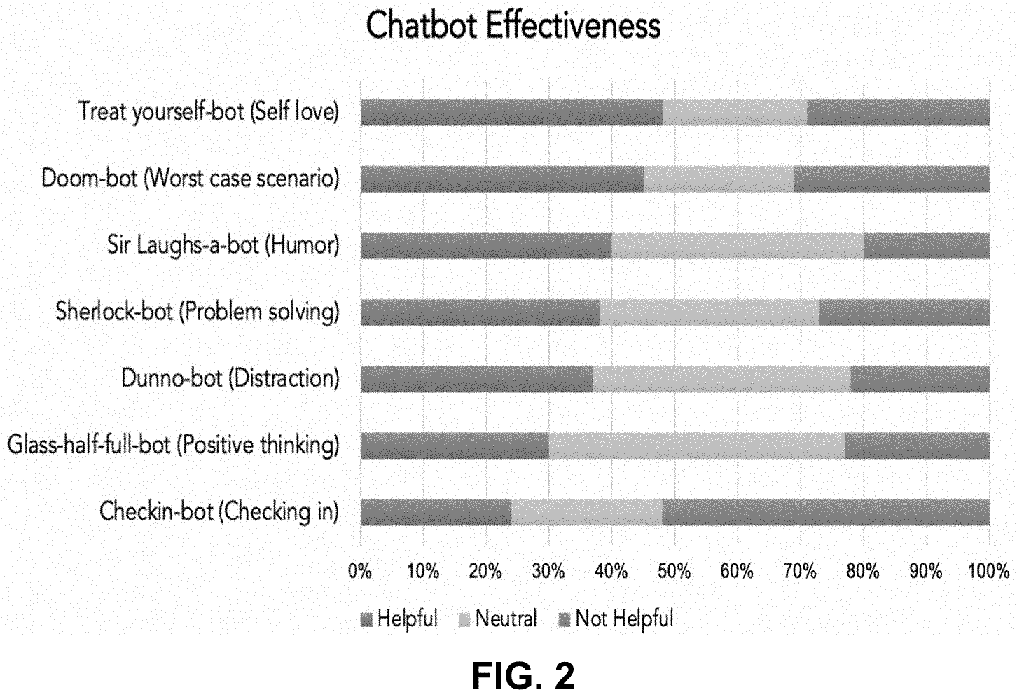

Overall, in-situ efficacy was either helpful ( 76/197, 39%) or neutral (64, 32%). While the remaining was rated as unhelpful (57, 29%). The inventor also observed that feedback varied by chatbot (FIG. 2). For example, nearly half of Treat-Yourself-bot's conversations were rated as helpful versus Check-in-bot's which were mostly viewed as unhelpful. The inventor believes this result is encouraging as it suggests that with more data patterns between stressors and chatbot or user and chatbots may emerge that might explain these differences and allow a future system to learn and make personalized recommendations.

### Daily Surveys

The daily survey was administered each evening at 8 pm (local time). In addition to usability questions, the survey tracked levels of stress, social interaction, and sleep quality the previous night using 5-point Likert scales rated None to Very High or Very Poor to Very Good. As a third ( 67/197) of conversations could not be matched to a daily survey (i.e., because the participants did not complete them that day), our analysis focuses on describing trends in the matched conversations (130).

### Stress Levels

The analysis of the daily surveys and conversational feedback indicates that most users experienced Low to Moderate levels of stress throughout the week and tended to rate the chatbots as Helpful. Moreover, the inventor observed that the chatbots seem to be less effective when participants reported higher levels of stress, though there were fewer such cases reported in the study.

### Sleep Quality

Similarly to daily stress, the data suggests that sleep quality might influence perceptions of interactions with our chatbots. Participants who reported Acceptable or better sleep quality the night before tended to report conversations as being Helpful.

### Social Interaction

Participants also reported Low to High levels of social interaction each day. Those who reported high levels of social interaction tended to be more positive about their interactions with the chatbots.

### Summary

Based on the daily survey data, participants appear to represent generally healthy people who report Acceptable or better levels of sleep quality and Low to High degrees of social interaction. As it is not uncommon for healthy people to experience stress-free days or days with Low to Moderate levels of stress, the fact that most participants rated daily conversations as Helpful to Neutral is promising given our general focus on this population.

### Post Study Experiential Feedback

Open-ended feedback from the post-study questionnaire was generally positive and helps to characterize the participant experience. For example, while it appeared from application logs that participants were using the chatbots throughout the day, most considered using the chatbots a private activity and, as a result, reported that they were difficult to use in the moment.

Most participants ( 28/31) reported using the chatbots when they were alone—typically when they had a free moment (i.e., a few hours after the stressful event). This was often because in work and social environments participants were busy or wanted to avoid giving the perception of rudeness caused by being on their phones which is an interesting potential barrier.

Like the in-situ conversational feedback, retrospective feedback on effectiveness skewed positive. Most ( 25/31) viewed the chatbots as Slightly Effective to Very Effective, about a quarter ( 9/31) described the chatbots as Not Effective at All. About half ( 17/31) described the current set of chatbots as cute and engaging. They also appreciated the concept of having a variety of chatbot options available. As P7596 explained “I like the ability to have access to different chatbots. I liked problem solving bot and check in bot, but the laugh bot not so much.” However, with only seven chatbots available, some (6) commented that their interactions with the chatbots felt formulaic and repetitive. Moreover, several (4) mentioned that it was difficult to remember the different names of the chatbots and what they were supposed to do. As P9329 described, “ . . . it would have been nice to know what each bot was supposed to be geared toward without having to engage each one.” A few (3) mentioned their preference for a single chatbot. One wrote “I would think a single bot that sensed the best approach would be more effective.” While most participants ( 22/31) were unlikely to continue to message with the chatbots in their current state, almost half (16) would recommend them to a friend, and one participant asked if they could continue to message with the chatbots after their participation post-study.

### Pre-Post Study Comparison

As part of the analysis the inventor looked at changes in several questions asked across the pre- and post-study questionnaires. These pre and post metrics include changes in PHQ-4 anxiety/depression scores, perceptions of daily stress, and perceptions of chatbots for stress management. To further explore differences, a post hoc analysis was conducted. Users were separated into two groups based on the number of conversations participants had with the chatbots. Specifically, the inventor grouped participants who completed less than one conversation with the system per day into the Low Usage group and those who had one or more conversations with the system per day into the High Usage group. Low Usage participants (N=16) had an average of 4.31 conversations over the course of the week (SD=1.31) whereas High Usage participants (N=15) had twice as many conversations (M=8.67, SD=2.12).

### PHQ-4

Overall, a decrease was observed in PHQ-4 scores when comparing pre and post assessments for participants who completed the study. A Wilcoxon signed-rank test showed that this difference was significant (P=0.01). However, the inventor cannot directly attribute this decrease to interactions with the chatbots without a control. However, the post hoc analysis suggests that participants in the Low Usage group reported lower before PHQ-4 scores compared to the High Usage group as well as a smaller difference in score reduction that was not significant. For participants in the Higher Usage group, the opposite was observed: higher before PHQ-4 scores and a greater difference in score reduction that was also significant (P=0.03).

### Daily Stress Experience

Perceptions of daily stress were evaluated using a 4-pt Likert scale rated A Little to A Great Deal. Though participants reported varying levels of stress on the daily survey, most described their perceptions of daily stress as Moderate in the pre study questionnaire and perceptions of daily stress during their participation were retrospectively similar. While a slight decrease in perceived daily stress was noticed, primary and post hoc analysis suggest these changes are not significant.

### Perceptions of Chatbots

Asked to describe their perceptions of chatbots for stress management on an open-response question, about half of participants ( 22/47) were neutral (i.e., stating the chatbots were interesting tools but not well developed), slightly more than a third (17) were positive (i.e., believing chatbots could be helpful), and the remaining (8) were negative (i.e., believing chatbots would not be effective or having no opinion on the topic). An illustrative comment in favor of chatbots was: “They seem to be a viable option for the management of stress, but they need to be further refined in order to be useful in day to day situations.” (P8530). In contrast, those who were more negative were best exemplified by P5219 who wrote: “ . . . it doesn't seem like talking to a non-human would be all that helpful because, for me, talking to a human doesn't usually help.”

However, in in the post study questionnaire most ( 20/31) participants reported a more positive attitude about chatbots for mental health. This was often because:
(i) they had a positive experience with the system themselves,
(ii) they could see such systems be helpful to people more generally, and/or
(iii) they found the activity of taking some time out each day to think about their stress helpful.

Additionally, about half ( 16/31) agreed that they had learned something about stress management from interacting with the system. For example, P8002 noted “I liked the idea of congratulating yourself for the things you did manage to do rather than focusing only on what you didn't”. Interestingly, even participants who did not report learning about stress management from the system were positive. For example, P9907 noted that while they did not learn anything from interactions with the chatbots they were “helpful reminders of what I should be doing when I am stressed” and others noted that while they did not learn anything directly from the chatbots they did learn that chatbots could be effective tool. Another third of participants (10) reported no change in their general attitudes about chatbots and a small number (3) reported a more negative attitude (i.e., finding the chatbots too repetitive).

### Follow-Up Card Sorting Interviews

As mentioned in the WoZ study, the interviews primarily centered around a card sorting activity with two phases. In the first phase, participants (N=13) were given 13 stressors to be assigned to the different chatbots based on which they felt were most effective. Stressors were synthesized from the Holmes and Rahe Stress Scale [40]. In the second phase of the activity, participants were asked to redistribute the stressor categories given three additional human options alongside chatbots: a non-trained stranger, friends & family, and a therapist. Participants were asked to “Think Aloud” while making their assignments. Some participants did not assign all categories to a chatbot and/or human. Some participants assigned a category to more than one chatbot and/or human. Where a participant assigned a category to more than one chatbot and/or human, the counts were normalized by the total to avoid over counting.

### Card Sorting Results

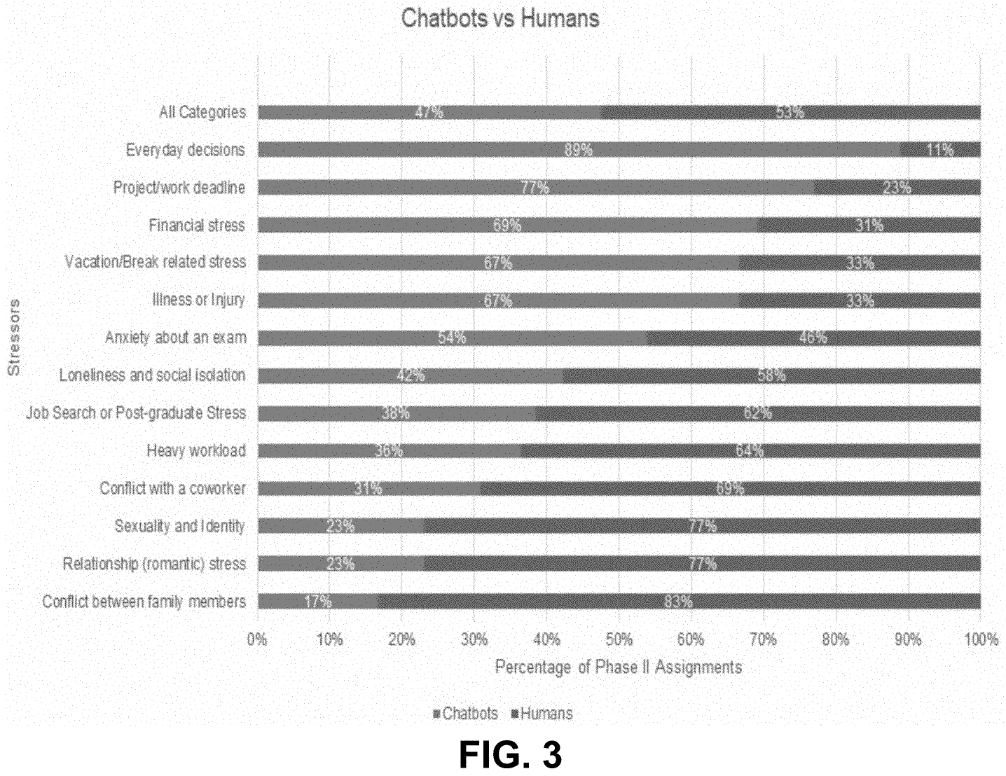

The card sorting activity suggests that there were certain stressors that participants preferred to talk to chatbots about given that not all assignments were reassigned in phase two when humans were available. The inventor observed that approximately 47% of stressor assignments were retained by the chatbots (FIG. 3). This result is critical and points toward a potential willingness of participants to use the chatbots for common daily stressors.

Moreover, when one sorts these stressors by those most assigned to chatbots one to observes that Everyday Decisions and Financial Stress were rarely reassigned to humans whereas interpersonal issues like Romantic Stress or Conflict with Family and complex topics like Sexuality and Identity were. However, not all chatbots performed equally well in terms of retaining their assignments in the presence of humans. For example, Table 4 indicates that Checkin-bot, Sherlock-bot, and Doom-bot were some of the more resilient chatbots whereas most of Dunno-bot's assignments were reassigned to humans. In fact, many chatbots retained more than half of their assignments. It was noted that participants had a strong preference for assigning problems to Friends and Family over Therapists with one assignment made to strangers.

TABLE 4
Stressor assignments by chatbot and human source.

| | | Phase 2: Chatbots |
|---|---|---|
| Resource | Phase 1: Chatbots | & Humans (Δ) |
| Sherlock-bot | 27% | −14% |
| Glass-half-full-bot | 18% | −15% |
| Doom-bot | 14% | −7% |
| Sir Laughs-a-bot | 13% | −7% |
| Treat Yourself-bot | 12% | −5% |
| Dunno-bot | 9% | −6% |
| Checkin-bot | 9% | −1% |
| Friends & Family | 0% | +35% |
| Therapist | 0% | +17% |
| Stranger | 0% | +1% |

### Qualitative Insights

As participants made their assignments of stressors to available chatbot and human resources, the inventor probed for their rationale. Overall, the inventor corroborated important themes around the desire to have chatbots that are part of an ecosystem of support supplementing humans, that behave in a human-like way, and are available to discuss certain stressors.

First Impressions. One challenge with chatbots is that of first impressions. About half of participants ( 6/13) thought their first interaction with a chatbot had an impact on their overall perceptions of the multiple chatbots available and an unpleasant first interaction with a chatbot left participants with a negative impression. As P1962 stated, “I went on the app and the bot said, ‘Find a joke’ and it was something actually really terrible that was going on. That was my first time interacting with the bots. I thought ‘Wow, there's nothing that's funny about this.’ This is not helpful at all.” (P1962)

Benefits of Multiple Chatbots. Participants described several benefits of having multiple chatbots available including the ability combine more than one chatbot to address a problem. This point was raised during the Wizard of Oz experiment by a participant who was insistent that problem-solving is ultimately the solution to all stressors, although other interventions may be used prior to, or in conjunction with, problem-solving for better results: “Everything is going to end up here in problem solving. If people are calm and collected, then they can think well. So, if people are calm first, then they will find everything's fine. Sometimes you can go from extreme stress to humor, but that's a big jump. I think it's better if you're slightly calmer and then humor comes in and then distraction.”—PB

This idea was further probed during the later phases of the study. Nearly half ( 6/13) of participants agreed that using multiple chatbots (or interventions) in combination could be an effective strategy to address stressors. Several participants were interested in using other interventions in combination with problem-solving: “In the case of conflict with a coworker, distracting yourself, not letting it take over your life, looking at the positive side of things could help. It could also go to the treat yourself And then the worst-case scenario, ‘Sure, I no longer interact with this coworker, and that's okay.’ In the end going back to the Problem Solving.”—P7

However, one participant (P9) noted that while more than one chatbot can be helpful to address a problem, it is not necessary to use them at the same time.

Talking with Friends & Family. Most participants ( 11/13) favored talking with friends & family over chatbots and they indicated that this preference had to do with the complexity of the stressor. Participants preferred speaking with friends and family about difficult emotional problems (e.g. conflicts with coworkers or interpersonal relationships). As P1442 summarized, “It depends on the degree of the problem. If it is a huge problem, I want a real person. If it's medium to small problem, then I go to the bot” (1442). There were several reasons for this preference including relationship history and range of responses. Friends and family already have pre-existing relationships with the participants and knowledge about their personal lives. About a third (4) preferred humans because of they can show empathy. Another third (4) believed that humans are better at problem-solving.

Talking with Therapists. Similar to talking with friends and family, more than half of participants ( 7/13) said that they believed therapists would be more helpful than chatbots in resolving complex problems. As PC observed, “Therapists are trained and objective. They are actual people. You can have complex conversations and get answers to questions with them” (PC). For example, nearly half ( 3/7) believed a therapist would be very helpful for talking through issues of sexual identity.

Talking with Chatbots. Participants noted several practical and emotion benefits to talking with chatbots. Regarding practical reasons, most participants ( 11/13) suggested there are some benefits to talking with chatbots compared to talking with humans. Almost half (5) mentioned that talking to a chatbot could help them avoid putting undue burden on others. As P7596 stated: “[Work stress] can be in the middle of the day, and [my friends] are going to be busy, and I don't want to text them and bother them about that” (P7596). Similarly, some (3) also noted that chatbots are easy to access. As P7596 described “It's going to be a lot quicker to pull up an app right? I sneak away to a room, I pull up the bot app, it's a lot quicker than messaging someone like, ‘Hey, are you around?’ and then waiting for a message back, or calling someone” (P7596). Another reason cited by a few (3) was that they could more easily control how much they told chatbots whereas humans are more likely to press for information.

Regarding emotional coping, participants described that the chatbots allow them to shift their focus. For example, more than half of participants ( 8/13) reported that Doom-bot helped them to re-calibrate the gravity of their stressor. As P7 described: “it's nice to hear when it feels like you're on the brink of doom, that like, oh, this is the worst thing that can happen” (P7). Similarly, half of participants (7) described the chatbots as distracting from their problems.

Interestingly, however, only 9% percent of stressors were assigned to the Dunno-bot (distraction), despite many people feeling that distraction could be an effective coping strategy. Almost half observed that humor helps them ameliorate their stress, one stated, “humor is often the antidote” (P7616). They noted that chatbots with a funny bone could be especially effective for stress management. Finally, a few (4) mentioned Glass-Half-Full-bot as being effective for putting stressful events in a different light. One participant imparted that reflecting on positive aspects of their experience allows them to, “take the edge off and make [the situation] work” (PD).

Privacy & Trust. When participants were asked about their privacy concerns while using the platform and to weigh the different privacy concerns, participants were split. About half ( 6/13) found some topics too personal to tell friends and family but were open to telling chatbots because of the perceived privacy they provide. For example, P1962 noted “I'm a very private person. I don't like to talk about a lot of things even with friends and family or in therapy” (P1962). Others went as far as to say that chatbots were more trustworthy because, as P7596 stated, they are “devoid of things that come with being human like judgement or telling secrets” (P7596). In contrast, a few (4) noted that they were aware that their messages were not private and took comfort in knowing that therapists were ethically bound to keep conversations confidential. The remaining (3) were unsure, as PD described “I don't know whether to worry about privacy or not. I think I have brand loyalty, so I always feel like Apple is gonna keep my stuff private” (PD).

When time allowed, the inventor probed a bit more on this topic to get a sense of how users felt about chatbot systems using their data to improve intervention efficacy. Two concerns emerged. First, about a third of participants ( 4/13) expressed concern about the utilization of conversational logs and other metadata that can be collected about online experiences. For example, P1962 likened such systems to other technology-related privacy incidents stating: “even though I found the chatbots helpful, if they were like [Amazon's] Alexa, running in the background waiting and listening to you and recording everything, I wouldn't like that” (P1962). Another quarter (3) were concerned that, even with additional training, chatbots might not be able to be trusted to handle mental health crises (e.g., referring users to proper resources). As 6716 summarized, “chatbots should potentially set off an alarm and say there needs to be a human to prevent this person from doing something terrible, as opposed to just being an ultra-safe communication cocoon” (P6716). In contrast, two were unconcerned about the handling of their data as long as it's used improved their experience. As P2 stated “I'm okay with chatbots having a lot of data about me if it's going to help them to respond better” (P2).

### Recommendation Systems

In this invention, chatbots were recommended to users at random. While conversational feedback was generally positive, some chatbots could perform better than others (i.e., feedback was more positive) and the inventor theorizes that installing a recommendation engine that can better match a shallow chatbot to the user's stressor could improve feedback further. An online reinforcement learning algorithm that can better take into account contextual, conversational, and prior interaction data would likely improve this matching between user problem and shallow chatbot intervention potentially even personalizing to the users' specific preferences over time.

### Design Recommendations

Based on this work, researchers and application designers designing multiple chatbots with a similar architecture might benefit from considering the following design recommendations: (i) focus on lowering barriers to authorship and generating numerous shallow chatbots based on the vast amount of available psychological interventions for stress management, (ii) design for online learning algorithms to handle recommendation and curation, (iii) attempt to score, rank, and classify daily stressors before assigning chatbots (interventions) toward accommodating differences in low and high complexity stressors as well as addressing concerns about identifying problems that are too severe for the system to handle, and (iv) consider a multitude of user coping styles, including those who may need a guided intervention or just an opportunity to reflect by talking or typing it out “into the void” quickly. If these problems can be addressed, then there is a real possibility to use this design paradigm to enable a new breed of shallow chatbot systems that might be more engaging over the long-term. However, the most difficult task is to convey the nature of these shallow chatbots to potential users. For the Popbots, the target group is healthy people undergoing regular stress who might be less likely to use daily preventative health systems. This group is a relatively understudied population in mental health, making research into engaging with them another important focus.

What is claimed is:

1. A method for management of daily stressors, comprising:
(a) having a plurality of digital services operating on a computer platform, wherein the plurality of digital services is displayed on a graphical computer user interface to a user, wherein each of the plurality of digital services uses natural language for interacting with the user, where each of the plurality of digital services is a script uniquely focusing on a single coping technique or type of intervention for daily stressors;
(b) selecting one or more digital services from the plurality of digital services for interfacing and interacting with the user via the graphical computer user interface, wherein the selected one or more digital services are related types of coping techniques or interventions that are based on an initial input received from the user via the graphical computer user interface; and
(c) conducting with the user via the computer user interface the open conversational exchange with the user based on one or more selected digital services.

2. The method as set forth in claim 1, wherein each script is a pre-scripted set of open conversational exchanges each containing 10 to 20 conversational exchanges and the script lasting about 2 to 3 minutes total.

3. The method as set forth in claim 1, wherein the plurality of digital services is displayed on the graphical computer user interface as multimedia elements including icons, images, text or a combination thereof.

4. The method as set forth in claim 1, wherein the step of selecting one or more digital services is done randomly by a software program running on the computer platform.

5. The method as set forth in claim 1, wherein the step of selecting one or more digital services is done randomly by a software program running on the computer platform based on a condition or user input.

6. The method as set forth in claim 1, wherein the user interacts with the graphical computer user interface by text, speech, or manipulating interface menus, buttons or multimedia interactive elements.

7. The method as set forth in claim 1, wherein the computer platform is a computer, a laptop, a smart phone, a computer tablet, a vehicle digital information system, a smart watch, a smart speaker, or an interactive computing device designed for user interactions.

8. The method as set forth in claim 1, wherein the computer platform is one or more computer servers or cloud services operating via an Internet protocol and communicating for user interaction with a computer, a laptop, a smart phone, a computer tablet, a vehicle digital information system, a smart watch, a smart speaker, or an interactive computing device designed for user interactions.

9. The method as set forth in claim 1, further comprising assigning and labeling a daily stressor based on the conversational exchange.

10. The method as set forth in claim 1, further comprising the user requesting a change in the selection of the one or more digital services.
